(19) United States
(12) Patent Application Publication
(10) Pub. No.: US 2010/0305015 A1
(43) Pub. Date: Dec. 2, 2010

(54) SKIN CLEANSING COMPOSITIONS

(75) Inventor: John Hawkins, South Staffordshire (GB)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(73) Assignee: INNOVATION DELI LIMITED, Lincolnshire (GB)

(21) Appl. No.: 12/446,379

(22) PCT Filed: Oct. 19, 2007
(86) PCT No.: PCT/GB07/04021

§ 371 Date: Jun. 19, 2009

(30) Foreign Application Priority Data
Oct. 20, 2006 (GB) 0620896.1
Dec. 21, 2006 (GB) 0625499.9
Oct. 11, 2007 (GB) 0720064.5

(51) Int. Cl. A61K 8/92; A61Q 19/10; A61K 8/20

(52) U.S. Cl. 510/139

(57) ABSTRACT

An exfoliant body scrub having, as exfoliant, solid salt suspended in saturated brine containing sufficient surfactant to form, with said brine, a structured, solid suspending system, and optionally an oil dispersed in said brine.

### PRIORITY CLAIM

The present application is a National Phase entry of PCT Application No. PCT/GB2007/004021, filed Oct. 19, 2007, which claims priority from Great Britain Patent Application 0620896.1, filed Oct. 20, 2006, Great Britain Patent Application No. 0625499.9, filed Dec. 21, 2006, and Great Britain Patent Application No. 0720064.5, filed Oct. 11, 2007, the disclosures of which are hereby incorporated by reference herein in their entirety.

### FIELD OF THE INVENTION

The invention relates to skin cleansing compositions for use in personal hygiene. In particular it relates to salt scrubs, which are useful as skin cleansing products, containing solid particles of salt as an exfoliant.

### BACKGROUND OF THE INVENTION

Salt scrubs typically comprise a paste of salt, a mild surfactant, fragrance and oil. They are rubbed onto the skin and then washed off, leaving the skin softened and cleansed. Such products are gaining increasing popularity. Sun and Parr (Toiletries and Cosmetics, Vol. 118, No. 6, June 2003) provide a review of scrub formulations.

Current salt scrub formulations have a number of disadvantages. They are stiff pastes, which cannot be poured or dispensed from conventional dispensers used for shower gels, shampoos and similar personal cleansing or shower products. On application to the skin much of the solid tends to fall off, creating wastage and mess, and effectively restricting use to the shower cubicle, where the spillages may give rise to a slip hazard. Moreover, being oil based they require a separate wash with a conventional soap or shower gel, in order to remove the oil. Sun and Parr (vs.) state on page 36 that because salt is water soluble its abrasive properties can only be utilised in non-aqueous formulations.

It has now been discovered that some or all of these problems may be overcome by suspending solid salt in a pourable aqueous medium comprising saturated brine and a structured surfactant.

The use of structured surfactant systems has hitherto been confined to suspending water-insoluble, or sparingly soluble, solids. Their use for suspending very water-soluble solids, such as sodium chloride has not hitherto been envisaged.

Structured surfactants generally comprise an L_{α}-phase, in which bilayers of surfactant are disposed with the hydrophobic “tail groups” of the surfactant on the inside and the hydrophilic “head groups” on the outside of the bilayer. The bilayers lie in a parallel or concentric arrangement, usually alternating with layers of an aqueous medium.

L_{α}-phases are sometimes referred to in the art as G-phases. They are commonly characterised by the textures observed under the polarising microscope and/or by small angle X-ray diffraction, which usually shows peaks indicative of lamellar symmetry, e.g. first, second and sometimes higher order peaks with a d-spacing in a simple integral ratio 1:2:3. The d-spacing is given by the formula 2π/Q, where Q is the momentum transfer vector.

Structured suspending systems typically comprise dispersed lamellar, spherulitic and/or expanded lamellar phases. Dispersed lamellar phases are two phase systems, in which domains of a lamellar phase are dispersed in, or interspersed with, an aqueous phase to form a gel. They are described in EP 0 086 614.

Spherulitic phases comprise spheroidal bodies, usually referred to in the art as spherulites, with an onion-like structure comprising concentric shells of surfactant. The spherulites usually have a diameter in the range 0.1 to 15 microns and are dispersed in an aqueous phase in the manner of a classical emulsion, but interacting to form a structured system. Spherulitic systems are described in more detail in EP 0 151 884.

The third type of structured system is the expanded L_{α}-phase, which is a single phase having a wider d-spacing than conventional L_{α}-phase. Conventional L_{α}-phases, contain 60 to 75% by weight surfactant and have a d-spacing of 4 to 7 nanometers. Attempts to suspend solids in such phases result in stiff pastes which are either non-pourable, unstable or both. Expanded L_{α}-phases have a d-spacing greater than 8, e.g. 10 to 100 nanometers. They may be prepared by adding electrolyte to aqueous surfactants at concentrations below those required to form a normal L_{α}-phase. Expanded L_{α}-phases are described in more detail in EP 0 530 708.

### SUMMARY OF THE INVENTION

It has now been found, contrary to the prejudice in the art exemplified by Sun and Parr, that stable exfoliant compositions can be obtained by suspending particulate, solid salt in saturated brine containing sufficient surfactant to form, in conjunction with said brine, a solid-supporting structured surfactant system, together, preferably with a minor proportion of an oil

The invention, therefore, provides the use as a skin cleanser of a stable, pourable or pasty exfoliant composition comprising, as exfoliant, solid particles of salt, said salt comprising sodium chloride, potassium chloride and/or magnesium chloride, said particles being suspended in a saturated aqueous solution of said salt, and sufficient surfactant to form, in conjunction with said solution, a solid-supporting structured surfactant system.

According to a second embodiment the invention provides an exfoliant salt scrub composition for use as a skin cleanser which comprises:
a) A saturated aqueous solution of salt, said salt comprising sodium chloride, potassium chloride and/or magnesium chloride;
b) Solid particles of said salt stably suspended in said solution;
c) Sufficient surfactant to form a stable, solid suspending structured system in said solution;
d) From 0.1 to 10% by weight of oil dispersed in said solution.

According to a third embodiment the invention provides an exfoliant salt scrub composition for use as a skin cleanser which comprises: the aforesaid components (a) and (b) and from 5 to 16% by weight of a surfactant mixture comprising from 60 to 90% by weight of said mixture of an alkyl 1 to 5 mole ethoxy sulphate and from 10 to 40% of a betaine.

Wherever the context permits, the term “salt” as used herein refers to sodium chloride, and mixtures of sodium chloride with potassium, calcium and/or magnesium chlorides and/or with minor proportions of alkali metal and/or alkaline earth metal iodides, bromides, and/or sulphates. In particular it includes purified sodium chloride, sea salt, rock salt and table salt. It also includes Dead Sea salt. The latter comprises a only a minor proportion of sodium chloride, together with high proportions of potassium and magnesium chlorides.

The term “structured system” as used herein means a composition comprising water, surfactant, and dissolved salt in an amount sufficient to interact with the surfactant to form a mesophase, or a dispersion of a mesophase in a continuous aqueous medium, and which has the ability to immobilise non-colloidal, water-insoluble particles, while the system is at rest, thereby forming a non-sedimenting, fluid or pasty suspension.

### DESCRIPTION OF THE INVENTION

In the following discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The salt may consist of a substantially pure industrial sodium chloride, table salt, or preferably sea salt, Dead Sea salt, or rock salt.

The surfactant is preferably a mild surfactant of the type commonly used in personal care formulations. It may comprise anionic, amphoteric, zwitterionic, non-ionic and/or cationic surfactants.

A preferred anionic surfactant comprises alkyl ether sulphate, which is preferably the product obtained by ethoxylating a natural fatty or synthetic alcohol with ethylene oxide, optionally stripping any unreacted alcohol, reacting the ethoxylated product with a sulphating agent and neutralising the resulting alkyl ether sulphuric acid with a base. The alcohol has an average of more than 8, preferably more than 10, more preferably more than 12, but less than 30, preferably less than 25, more preferably less than 20, most preferably less than 15 carbon atoms. It is reacted with an average of at least 0.5, preferably more than 1, but less than 10, preferably less than 5, more preferably less than 4, most preferably less than 3 ethyleneoxy groups.

The anionic surfactant may also comprise alkyl glyceryl sulphates, and random or block copolymerised alkyl ethoxy/propoxy sulphates, C₁₀₋₂₀ e.g. C₁₂₋₁₈ alkyl sulphates, C₁₀₋₂₀ alkyl benzene sulphonates or C₈₋₂₀ e.g. C₁₀₋₂₀ aliphatic soaps. The soap may be saturated or unsaturated, straight or branched chain. The surfactant may also include other anionic surfactants, such as olefin sulphonates, paraffin sulphonates, taurides, isethionates, ether sulphonates, ether carboxylates, sarcosinates, aliphatic ester sulphonates e.g. alkyl glyceryl sulphonates, sulphosuccinates or sulphosuccinamates. The cation of any anionic surfactant is typically sodium but may alternatively be potassium, lithium, calcium, magnesium, ammonium, or an alkyl or hydroxyalkyl ammonium having up to 6 aliphatic carbon atoms including ethylammonium, isopropylammonium, monoethanolammonium, diethanolammonium, and triethanolammonium.

Ammonium and ethanolammonium salts are generally more soluble than the sodium salts. Mixtures of the above cations may be used.

The surfactant preferably comprises an amphoteric or more preferably a zwitterionic surfactant. The zwitterionic surfactant is preferably a betaine, phosphobetaine or sulphobetaine, which typically has the formula R″R′₂ NCH₂XOH, where X is CO, PO or SO₂, R′ is an aliphatic group having 1 to 4 carbon atoms and R″ is an aliphatic group having from 8 to 25 carbon atoms, preferably a straight or branched chain alkyl or alkenyl group, or more preferably a group of the formula RCONR′(CH₂)ₙ, where R and R′ have the same significance as before, and n is an integer from 2 to 4.

We prefer that R′ is a methyl, carboxymethyl, ethyl, hydroxyethyl, carboxyethyl, propyl, isopropyl, hydroxypropyl, carboxypropyl, butyl, isobutyl or hydroxybutyl group.

The amphoteric surfactant may comprise so-called imidazoline betaines, which are also called amphoacetates, obtained by reacting sodium chloracetate with an imidazoline.

The non-ionic surfactants may typically comprise amine oxides, polyglyceryl fatty esters, fatty acid ethoxylates, fatty acid monoalkanolamides, fatty acid dialkanolamides, fatty acid alkanolamide ethoxylates, propylene glycol monoesters, fatty alcohol propoxylates, alcohol ethoxylates, alkyl phenol ethoxylates, fatty amine alkoxylates and fatty acid glyceryl ester ethoxylates. Other non-ionic compounds suitable for inclusion in compositions of the present invention include mixed ethylene oxide/propylene oxide block copolymers, ethylene glycol monoesters, glyceryl esters, ethoxylated glyceryl esters, alkyl polyglycosides, alkyl sugar esters including alkyl sucrose esters and alkyl oligosaccharide esters, sorbitan esters, ethoxylated sorbitan esters, alkyl capped polyvinyl alcohol and alkyl capped polyvinyl pyrrolidone. Particularly preferred non-ionic surfactants include sugar esters and alkyl polyglycosides, such as C₁₀₋₂₀, preferably C₁₀₋₁₈, most preferably C₁₂₋₁₆ alkyl polyglucoside, preferably with a degree of polymerisation between 1.2 and 3.

The surfactant may comprise cationic surfactants such as fatty alkyl trimethylammonium or benzalkonium salts, amidoamines or imidazolines.

The surfactants preferably have a mean HLB greater than 10, more preferably greater than 15, even more preferably greater than 20, more preferably still, greater than 30, most preferably greater than 40. Difficulty may be encountered obtaining stable suspensions with surfactant systems having a mean HLB greater than 65. Preferably the HLB is less than 60, more preferably less than 55, most preferably less than 50.

The aqueous structured systems, formed by the interaction of surfactants with salt according to our invention typically comprise systems, which are either spherulitic or expanded L_{α}-phase. They include systems having a repeat spacing greater than 8, preferably greater than 10, more preferably greater than 20, most preferably greater than 30 nm, up to or above 60 nm.

We generally prefer that the surfactant is present in a total concentration greater than 4% by weight, based on the total weight of the composition, more preferably greater than 5%, still more preferably greater than 7%, even more preferably more than 8%, most preferably greater than 9%. Preferably the surfactant concentration is less than 20%, more preferably less than 17%, still more preferably less than 15% most preferably less than 14% by weight.

Preferably the surfactant comprises at least 50% by weight thereof, of an alkyl ether sulphate, more preferably at least 60%, most preferably at least 70%. Preferably the proportion of ether sulphate is less than 95%, more preferably less than 90%, most preferably less than 80% of the total weight of surfactant. Preferably the surfactant comprises at least 5%, by weight thereof, more preferably at least 10%, most preferably at least 20% of a betaine. Preferably the proportion of betaine is less than 50%, more preferably less than 40% most preferably less than 30% of the total weight of surfactant.

The discussion is based on the assumption that the structure is lamellar or spherulitic. We do not, however, intend to exclude the possibility that the system may comprise non-lamellar components.

The levels of salt may be sufficiently high to inhibit microbiological growth in the medium and sufficient to act as an effective biodegradable, non-allergenic preservative for the composition.

Preferably the surfactant is stirred into the saturated salt solution, and if a sufficiently stable suspending system is not obtained, low HLB surfactant is added in small increments until an acceptable yield point is achieved.

The solid exfoliant is present in total concentrations greater than saturation at ambient temperature. The composition generally comprises suspended solid salt in amounts, at room temperature, greater than 3%, preferably greater than 15%, more preferably greater than 25%, even more preferably greater than 35%, most preferably greater than 45% by weight, based on the weight of the composition. Amounts of suspended solid greater than 70% by weight are usually undesirably viscous. We prefer that the suspended solid should be less than 60% by weight, more preferably less than 65%

The suspended solid salt typically has a relatively coarse granular texture, with a mean particle size greater than 100 microns, preferably greater than 500 microns, more preferably greater than 1 mm, most preferably greater than 1.5 mm, but less than 5 mm, preferably less than 3 mm, most preferably less than 2 mm.

The composition preferably comprises an oil. In particular we prefer that the oil is a non-volatile oil such as a hydrocarbon, glyceride, silicone or other substantially water-immiscible liquid having a boiling point greater than that of water. We particularly prefer vegetable oils such as olive oil, yangu oil, jojoba oil, shea butter or peppermint oil.

The product may optionally contain other common ingredients of personal cleansers, such as buffers, antioxidants, glycerol, essential oils, fragrances, pigments, dyes, pearlisers, emollients, antiseptics and topical medicaments.

Buffers may be required to obtain optimum pH for stability of the ingredients and/or skin sensitivity. We prefer that the pH is less than 8, more preferably less than 7, most preferably less than 5.8, but more than 4, more preferably more than 5, most preferably more than 5.2. Suitable buffers, depending on the desired pH include citrate (e.g. trisodium citrate/citric acid), acetate, phosphate and tartrate buffers.

The product may be a readily pourable fluid, or a paste. Typically the viscosity at 21 reciprocal seconds shear is greater than 0.1 Pa s, more preferably greater than 1 Pa s, most preferably greater than 5 Pa s, but less than 25 Pa s, more preferably less than 20 Pa s, most preferably less than 15 Pa s.

The invention will be illustrated by the following examples, in which all proportions are expressed as % by weight based on the total weight of the composition unless stated to the contrary.

Each of the following examples I to VI was a stable, pourable spherulitic suspension with a pleasant feel when rubbed on the skin and an effective cleansing and skin softening action. In each case the balance was water, to which ingredients were added cold, with stirring, in the stated order.

### Example I

| | |
|---|---|
| Sodium lauryl 2-mole ethoxy sulphate. | 7.88 |
| Coconut amidopropyl betaine | 1.96 |
| Pure vacuum dried sodium chloride | 57.20 |
| Yangu oil | 1.97 |
| Perfume | 0.20 |
| Viscosity at 21 s⁻¹ | 1.2 Ps |

### Example II

| | |
|---|---|
| Sodium lauryl 2-mole ethoxy sulphate | 10.9 |
| Coconut amidopropyl betaine | 2.7 |
| Pure vacuum dried sodium chloride | 63.2 |
| Olive oil | 1.0 |
| Viscosity at 21 s⁻¹ | 6 Ps |

### Example III

| | |
|---|---|
| Sodium lauryl 2-mole ethoxy sulphate | 7.86 |
| Coconut amidopropyl betaine | 2.36 |
| Coarse sea salt | 59.00 |
| Peppermint oil | 0.30 |
| Olive oil | 1.97 |
| Viscosity at 21 s⁻¹ | 2 Ps |

### Example IV

| | |
|---|---|
| Sodium lauryl 2-mole ethoxy sulphate | 8.0 |
| Coconut amidopropyl betaine | 2.0 |
| Coarse sea salt | 58.0 |
| Perfume | 0.2 |

### Example V

| | |
|---|---|
| Sodium lauryl 2-mole ethoxy sulphate | 8.0 |
| Coconut amidopropyl betaine | 2.0 |
| Cooking salt | 58.0 |
| Perfume | 0.2 |

### Example VI

| | |
|---|---|
| Sodium lauryl 2-mole ethoxy sulphate | 7.0 |
| Magnesium lauryl 2-mole ethoxy sulphate | 7.0 |
| Dead Sea salt | 70.0 |
| Glycerol | 0.2 |

1. A stable exfoliant composition for use as apourable or pasty skin cleaner comprising:
an exfoliant having solid particles of salt, said salt comprising sodium chloride, potassium chloride and/or magnesium chloride, said particles being suspended in a saturated aqueous solution of said salt; and
a surfactant in an amount sufficient to form a solid-supporting structured surfactant system in conjunction with said saturated aqueous solution of said salt.

2. An exfoliant salt scrub composition for use as a skin cleanser which comprises:
a saturated aqueous solution of salt, said salt comprising sodium chloride, potassium chloride and/or magnesium chloride;
solid particles of said salt stably suspended in said solution;
sufficient surfactant to form a stable, solid suspending structured system in said solution; and
an oil dispersed in said solution in an amount from 0.1 to 10% by weight.

3. A composition according to claim 2 wherein said salt consists of a substantially pure sodium chloride, table salt, sea salt, Dead Sea salt, or rock salt.

4. A composition according to either of claim 2 wherein the surfactant comprises an alkyl ether sulphate.

5. A composition according to claim 4 wherein the surfactant additionally comprises a betaine.

6. A composition according to claim 2 wherein the oil is a hydrocarbon, glyceride or silicone oil.

7. A composition for use as an exfoliating skin cleanser comprising solid sodium chloride suspended in saturated brine, structured with sodium and/or ammonium lauryl 2 mole ethoxy sulphate.
